(19) United States
(12) Patent Application Publication
(10) Pub. No.: US 2014/0186321 A1
(43) Pub. Date: Jul. 3, 2014

(54) Method for Treating Metabolic Syndrome and Diabetes Using Quercetin and Bauhinia fortificata Extract

(71) Applicant: Thomas Christian Lines, Wayland, MA (US)

(72) Inventor: Thomas Christian Lines, Wayland, MA (US)

(21) Appl. No.: 13/729,544

(22) Filed: Dec. 28, 2012

(51) Int. Cl. A61K 36/48; A61K 31/353

(52) U.S. Cl. CPC A61K 36/48; A61K 31/353
USPC 424/94.1; 424/757; 514/690

(57) ABSTRACT

This invention relates to a method of treating metabolic syndrome and diabetes using a composition containing quercetin, vitamin B₃, vitamin C, and a Bauhinia fortificata extract.

### BACKGROUND

Metabolic syndrome is defined by a number of risk factors that, when they are present together in humans, double the risk of blood vessel and heart disease, and increase the risk of diabetes by five times. These risk factors include abdominal fat, unhealthy cholesterol levels, high blood pressure, and high or irregular levels of blood sugar.

More specifically, according to the American Heart Association and the National Heart, Lung, and Blood Institute, the following five risk factors define metabolic syndrome: I) Large waist size, defined as 40 inches or larger for men and 35 inches or larger for women; II) High triglycerides, defined as 150 mg/dL or higher; III) Low HDL cholesterol, defined as less than 40 mg/dL for men and less than 50 mg/dL for women; IV) High blood pressure, defined as at least 130/85 mm Hg; and V) High blood sugar, defined as a fasting glucose level of 100 mg/dL or higher.

Diabetes is a disease that results from either the body's inability to make insulin or by the body's inability to respond to insulin. Type 1 diabetes results from a loss of insulin-producing cells in the pancreas. The lack of insulin leads to high levels of glucose in the circulation. Type 2 diabetes develops when the body fails to respond properly to secreted insulin. This is known as insulin resistance, and, similar to type 1 diabetes, results in an increase in blood glucose levels.

Diabetes increases the risk of developing other serious medical problems. Such medical problems include, e.g., neuropathy necessitating amputations, retinopathy resulting in blindness, nephropathy leading to chronic kidney failure, heart disease, stroke, peripheral artery disease, and gum disease.

Typically, patients suffering from type 1 diabetes take insulin injections when necessary to maintain their blood glucose level at a particular value. Type 2 diabetes can be controlled by behavior modification, i.e., improving diet and exercising more, and can be treated using drugs that have been developed to lower blood glucose levels, including sulfonylureas, meglitinides, biguanides, thiazolidinediones, alpha-glucosidase inhibitors, DPP-4 inhibitors and adjunct therapies.

Quercetin, a natural antioxidant, is known to inhibit both acute and chronic phases of free-radical induced diseases such as disorders associated with C-reactive protein, cancer, and autoimmune disease. It is also known to enhance or maintain physical or mental performance, reduce infection exacerbated by physical stress, and improve immune system recovery from intense physical exercise.

Bauhinia fortificata extract is known to act as a hypoglycemic agent and has been used as an herbal treatment to lower blood sugar levels in diabetic animals and humans.

### SUMMARY

The present invention features a method for treating metabolic syndrome or diabetes by administering to a subject in need thereof an effective amount of a quercetin-containing composition, which also includes vitamin B₃, vitamin C, and a Bauhinia fortificata extract. In one example, the composition contains quercetin, vitamin B₃, vitamin C, and a Bauhinia fortificata extract at a weight ratio of 1:0.02-1.5:0.2-2.5:0.5-3.

The invention also features a method for improving the efficacy of a diabetes medication, in which an effective amount of the above-mentioned composition is administered to a subject taking a diabetes medication.

Also provided is a method for treating diabetes by administering to a subject in need thereof an effective amount of a composition that includes quercetin, vitamin B3, vitamin C, a Bauhinia fortificata extract, luteolin, and epigallocatechin gallate (EGCG) as the only active ingredients. An exemplary composition contains quercetin, vitamin B₃, vitamin C, and a Bauhinia fortificata extract at a weight ratio of 1:0.02-1.5:0.2-2.5:0.5-3.

The composition used in the above-described treatment, either in dry form (e.g., powder or tablet) or in liquid form (e.g., beverage or syrup), can be a dietary supplement or a pharmaceutical formulation, both of which can be in the form of a tablet, a capsule, a soft chew, or a gel. The composition can also be a food product, including tea (e.g., a tea drink and the contents of a tea bag), soft drink, juice (e.g., a fruit extract and a juice drink), milk, coffee, jelly, ice cream, yogurt, cookie, cereal, chocolate, and snack bar.

The details of one or more embodiments of the invention are set forth in the description below. Other features, objects, and advantages of the invention will be apparent from the description and from the claims. All references cited herein are hereby incorporated by reference in their entirety.

### DETAILED DESCRIPTION

The present invention is based on the unexpected discovery that administering a composition containing quercetin, vitamin B₃, vitamin C, and a Bauhinia fortificata extract to a subject with metabolic syndrome or diabetes, results in a lowering of blood glucose levels and blood pressure, and an increase in blood flow and insulin sensitivity, all indications of an improvement in metabolic syndrome or the diabetic condition.

Accordingly, the present invention features a method for treating metabolic syndrome and diabetes by administering an effective amount of a composition containing quercetin, vitamin B₃, vitamin C, and a Bauhinia fortificata extract.

Without being bound by theory, quercetin, vitamin B₃, and vitamin C, combined with Bauhinia fortificata extract, may function to ameliorate diabetes and metabolic syndrome via the following mechanisms.

Prolonged daily use of quercetin serves to improve carbohydrate and protein metabolism. It also leads to an increase in the production of bile acids that facilitate absorption of fats in the intestines and excretion of fats and sterols in the liver. Bile acids are also essential for dietary fat-based vitamin absorption. Quercetin also causes an increase in the level of palmitic acid, which ultimately lowers insulin sensitivity. This property of quercetin can allow for a composition containing it to maintain a constant blood sugar level. Further, quercetin increases the level of stearic acid, leading to an improved lipid profile and a reduced risk of developing cardiovascular disease in diabetics. Additionally, quercetin beneficially increases the glomerular filtration rate in diabetics.

The efficacy of quercetin is enhanced by vitamin B₃ and vitamin C. More specifically, a combination of quercetin, vitamin B₃, and vitamin C maintains quercetin levels in plasma up to five times those of quercetin alone or a combination of quercetin and vitamin B₃; and that a combination of quercetin, vitamin B₃, and vitamin C results in a quercetin half life in plasma twice as long as that of quercetin alone and about one and a half times that of a combination of quercetin and vitamin B3. See U.S. Pat. Nos. 7,745,486 and 7,745,487.

It is disclosed that quercetin and Bauhinia fortificata extract together have a synergistic effect. The combination of quercetin and Bauhinia fortificata extract increases blood flow more than either individually, greatly reducing the risk of thrombotic events in diabetics. The quercetin/Bauhinia fortificata extract combination also lowers blood pressure to a greater degree than either agent alone. The combination of quercetin and Bauhinia fortificata extract also synergistically increases insulin sensitivity in type 2 diabetics.

The invention also features a method for improving the efficacy of a diabetes medication, in which an effective amount of the above-mentioned composition is administered to a subject taking a diabetes medication. In one aspect, improving the efficacy of a diabetes medication according to the present invention will allow for a subject to be treated with lower doses of a diabetes medication. For example, a diabetes patient being treated with 40 units of insulin daily can be treated with a lower dose (e.g., 30 units, 20 units, 10 units, or less) if concurrently administered one of the above-described compositions. Similarly, a patient taking 40 mg of glipizide daily can reduce that amount to 30 mg, 20 mg, 10 mg, 5 mg, 2.5 mg, or less if a composition of the current invention is co-administered.

The ability to treat a subject using lower doses of a diabetes medication can, for example, advantageously reduce the side effects associated with the diabetes medication. Such side effects include, but are not limited to weight gain, nausea, low blood glucose, diarrhea, gas, loss of appetite, liver damage, fluid retention, and stomach pain.

Co-administration refers to administering the diabetes medication and the inventive composition at the same time or at different times. For example, a diabetes medication can be administered in one daily dose, while the inventive composition can be administered four times daily. Alternatively, the diabetes medication and the composition of the invention can both be administered prior to a meal.

A diabetes medication mentioned herein can be, but is not limited to, a sulfonylurea (glimepiride, glipizide, glyburide), a meglitinide (repaglinide, nateglinide), a biguanide (metformin), a thiazolidinedione (pioglitazone, rosiglitazone), an alpha-glucosidase inhibitor (acarbose, miglitol), a DPP-4 inhibitor (sitagliptin, saxagliptin, vildagliptin, linagliptin), or an adjunct therapy (exenatide, pramlintide).

The term “quercetin” refers to both quercetin aglycon and quercetin derivatives, e.g., quercetin-3-O-glucoside (isoquercetin), quercetin-5-O-glucoside, quercetin-7-O-glucoside, quercetin-9-O-glucoside, quercetin-3-O-rutinoside, quercetin-3-O-[α-rhamnosyl-(1→2)-α-rhamnosyl-(1→6)]-β-glucoside, quercetin-3-O-galactoside, quercetin-7-O-galactoside, quercetin-3-O-rhamnoside, and quercetin-7-O-galactoside. After digestion, quercetin derivatives are converted to quercetin aglycon and other active derivatives, which are absorbed in the body.

A Bauhinia fortificata extract mentioned herein refers to an extract typically obtained by extracting parts of a Bauhinia plant, e.g., leaves, with a mixture of polar solvents. For example, a mixture of polar solvents may contain water as one solvent and at least one other solvent that is less polar than water (e.g., methanol, ethanol, 1-propanol, 2-propanol, acetonitrile, acetone, and ethyl acetate). The extraction is typically carried out at a temperature between 20° C. and 90° C. The resulting extract can be concentrated and the concentrate can be dried, if preferred.

In one preferred embodiment, a composition including isoquercetin, vitamin B₃, vitamin C, and a Bauhinia fortificata extract is co-administered with metformin to control diabetes.

An exemplary Bauhinia fortificata extraction protocol follows. 100-200 kg of ground Bauhinia fortificata leaves is added to a vessel containing 1000-2000 L of 50% ethanol in water and incubated at 70-75° C. under reflux for 60-75 min. The resultant slurry is filtered under pressure successively through a filter press, a lenticular filter, a 24-disc sparkler filter, and a 0.5 μM filter. The solids removed from the slurry are re-extracted with 50% ethanol in water as described above. The re-extraction liquid is filtered as described above. The two filtrates are combined and loaded into a vacuum reactor in 1000 L aliquots, after which each aliquot is concentrated to a volume of 200-300 L. The concentration of solids in the resultant purified extract is 18-20 w/v %. The purified extract can be further evaporated as required for a particular formulation.

The term “treating” refers to the administration of an effective amount of a composition containing quercetin, vitamin B₃, vitamin C, and a Bauhinia fortificata extract to a subject with diabetes or metabolic syndrome with the purpose of improving one or more of the conditions/symptoms accompanied with elevated or irregular levels of blood sugar, elevated serum cholesterol levels, or elevated blood pressure, or to prevent, cure, alleviate, relieve, remedy, or ameliorate one or more of the conditions/symptoms, or the predispositions to one or more of them. In one preferred embodiment, the composition includes isoquercetin, vitamin B₃, vitamin C, and a Bauhinia fortificata extract.

An example of a composition for practicing this invention includes quercetin, vitamin B₃, vitamin C, and a Bauhinia fortificata extract, the weight ratio of which in the composition can be 1:0.02-1.5:0.2-2.5:0.5-3, or any ratio in between. For example, the weight ratio can be 1:0.04-0.5:0.3-2.0:0.5-3, 1:0.05-0.3:0.4-1.5:0.5-3, 1:0.05-0.2:0.5-1:0.5-3, and 1:0.1-0.2:0.5-1:0.5-3. Preferred ratios include about 1:0.04:0.5:1.5, about 1:0.04:1:1, about 1:0.04:0.5:0.5, and about 1:0.5:0.5:1.

Typically, a subject can be administered, once or periodically per day, with an effective amount of the composition, e.g., in an amount that provides 100 mg to 2 g (preferably, 250 mg to 1 g) of quercetin and 50 mg to 6 g (preferably, 125 mg to 0.5 g) of Bauhinia fortificata extract.

The quantity of quercetin mentioned above refers to that of quercetin aglycon or the quercetin moiety of a quercetin derivative such as isoquercetin. Quercetin can be added to the composition either in a pure form or as an ingredient in a mixture (e.g., a plant extract). Examples of commercially available quercetin include QU995 (containing 99.5% quercetin) and QU985 (containing 98.5% quercetin) from Quercegen Pharma LLC (Newton, Mass.) and Merck KGaA (Brazil). “Vitamin B₃” mentioned herein includes vitamin B₃ in its various forms, including niacinamide, nicotinic acid, nicotinamide, and inositol hexaniacinate. “Vitamin C” mentioned herein includes vitamin C (i.e., L-ascorbic acid, D-ascorbic acid, or both) and its salts (e.g., sodium ascorbate).

The term “administration” covers oral or parenteral delivery to a subject a composition of the invention in any suitable form, e.g., food product, beverage, tablet, capsule, suspension, and solution. The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques. An “effective amount” refers to a dose of the composition that is sufficient to provide a therapeutic benefit (e.g., lowering or stabilizing blood sugar levels). Both in vivo and in vitro studies can be conducted to determine optimal administration routes and doses.

The composition used in the method of this invention can be in various forms. For example, it can be a soft chew composition that includes quercetin, niacinamide, ascorbic acid, sodium ascorbate, Bauhinia fortificata extract, sugar, corn syrup, sucralose, soy lecithin, corn starch, clycerin, palm oil, xylitol, carrageenan, FD&C Yellow #6, FD&C Yellow #5, and natural and/or artificial flavors. An exemplary serving of this soft chew composition (5.15 g) includes 250 mg of quercetin, 12.9 mg of vitamin B₃ (i.e., niacinamide), 382.8 mg vitamin C (i.e., L-ascorbic acid and sodium ascorbate) and 125 mg of Bauhinia fortificata extract. A subject can take one to eight servings (e.g., 4 servings) of this soft chew composition daily. The amounts taken can vary depending on, for example, the disorder or condition to be treated and the physical states of the subject. Another exemplary composition of this soft chew includes 5.25 wt % of quercetin, 0.25 wt % of vitamin B3, 7.81 wt % of vitamin C (i.e., L-ascorbic acid and sodium ascorbate) and 2.75 wt % of Bauhinia fortificata extract.

The composition can further contain one or more of active ingredients, such as folic acid (including vitamin B9, folate, pteroylglutamic acid, and their derivatives, e.g., methylfolate), an isoflavone (e.g., genistein or genistin), curcumin, resveratrol, luteolin, epigallocatechin gallate (EGCG), coenzyme Q10, eicosapentaenoic acid (EPA), and docosahexaenoic acid (DHA). These active ingredients can be added to the composition either in a pure form or as a component in a mixture (e.g., an extract from a plant or an animal). A suitable daily dosage of each of these ingredients can vary depending on, for example, the disorder or condition to be treated and the physical states of the subjects. Exemplary daily dosages of some of these ingredients are: 400-800 mcg of folic acid, 20-2,500 mg (preferably 250-1,000 mg) of curcumin, 10-1,000 mg (preferably 100-500 mg) of resveratrol, 50-1,000 mg (preferably 100-700 mg) of EGCG, 25-300 mg (preferably 50-100 mg) of genistin/genistein, 10-1,000 mg (preferably 100-200 mg) of luteolin, 50-1,000 mg (preferably 70-500 mg) of EPA, and 50-1,000 mg (preferably 80-700 mg) of DHA. Further, it can be sweetened, if necessary, by adding a sweetener such as sorbitol, maltitol, hydrogenated glucose syrup and hydrogenated starch hydrolyzate, high fructose corn syrup, cane sugar, beet sugar, pectin, and sucralose. The composition can also contain amino acids, fatty acids, proteins, fibers, minerals, a flavor enhancer, or a coloring agent. Exemplary amino acids include theanine (e.g., L-theanine) and alanine (e.g., L-alanine). Exemplary fatty acids include omega-3 fatty acids (e.g., linolenic acid), omega-6 fatty acids (e.g., linoleic acid), and omega-9 fatty acids (e.g., oleic acid). Exemplary proteins include plant proteins, such as soy proteins and chia seed proteins. Exemplary fibers include plant fibers, such as soy fibers and chia seed fibers. These ingredients can be added in the above-described composition either in a pure form or as a component in a mixture (e.g., an extract from a plant or an animal).

In another embodiment, a composition useful for treating metabolic syndrome and diabetes includes quercetin, vitamin B3, vitamin C, a Bauhinia fortificata extract, luteolin, and epigallocatechin gallate (EGCG) as the only active ingredients, and the weight ratio between quercetin, vitamin B3, vitamin C, and the Bauhinia fortificata extract is 1:0.02-1.5:0.2-2.5:0.5-3.

When the above-described composition is in powder form, it can be used conveniently to prepare beverage, paste, jelly, capsules, or tablets. Lactose and corn starch are commonly used as diluents for capsules and as carriers for tablets. Lubricating agents, such as magnesium stearate, are typically included in tablets.

The composition of this invention can be a dietary supplement or a pharmaceutical formulation. As a dietary supplement, additional nutrients, such as minerals or amino acids may be included. The composition can also be a food product. As used herein, the term “food” broadly refers to any kinds of liquid and solid/semi-solid materials that are used for nourishing humans and animals, for sustaining normal or accelerated growth, or for maintaining stamina or alertness. Examples of human food products include, but are not limited to, tea-based beverages, juice, coffee, milk, jelly, cookies, cereals, chocolates, snack bars, herbal extracts, dairy products (e.g., ice cream, and yogurt), soy bean product (e.g., tofu), and rice products.

The compositions described above can be preliminarily screened for their efficacy in treating the above-described conditions in animal models and in clinic trials. For example, the effectiveness of the above described compositions can be evaluated by conducting a glucose tolerance test following administration of the composition to a subject. Other suitable analytical and biological assays are apparent to those of ordinary skill in the art.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A composition for treating metabolic syndrome or diabetes, the composition comprising quercetin, vitamin B3, vitamin C, and a Bauhinia fortificata extract, wherein a weight ratio between quercetin, vitamin B3, vitamin C, and the Bauhinia fortificata extract is 1:0.02-1.5:0.2-2.5:0.5-3.

2. The composition of claim 1, wherein the weight ratio is 1:0.04:1:1.

3. The composition of claim 1, wherein the weight ratio is 1:0.04:0.5:0.5.

4. The composition of claim 1, wherein the weight ratio is 1:0.5:0.5:1.

5. The composition of claim 1, wherein the composition is in dry form.

6. The composition of claim 1, wherein the composition is in liquid form.

7. The composition of claim 1, wherein the composition is a food product.

8. The composition of claim 1, wherein the composition is a dietary supplement or a pharmaceutical formulation.

9. The composition of claim 8, wherein the composition is a tablet, a capsule, a soft chew, or a gel.

10. The composition of claim 1, further comprising folic acid, genistein, genistin, curcumin, resveratrol, luteolin, epigallocatechin gallate (EGCG), coenzyme Q10, eicosapentaenoic acid (EPA), or docosahexaenoic acid (DHA).

11. The composition of claim 10, wherein the composition includes luteolin or epigallocatechin gallate (EGCG).

12. A method for treating diabetes, the method comprising administering to a subject in need thereof an effective amount of a composition that includes quercetin, vitamin B3, vitamin C, and a Bauhinia fortificata extract, wherein a weight ratio between quercetin, vitamin B3, vitamin C, and the Bauhinia fortificata extract is 1:0.02-1.5:0.2-2.5:0.5-3.

13. The method of claim 12, wherein the composition further includes folic acid, genistein, genistin, curcumin, resveratrol, isoquercetin, luteolin, epigallocatechin gallate (EGCG), coenzyme Q10, eicosapentaenoic acid (EPA), or docosahexaenoic acid (DHA).

14. A method for improving the efficacy of a diabetes medication, comprising administering to a subject taking a diabetes medication an effective amount of a composition that includes quercetin, vitamin B3, vitamin C, and a Bauhinia fortificata extract, wherein a weight ratio between quercetin, vitamin B3, vitamin C, and the Bauhinia fortificata extract is 1:0.02-1.5:0.2-2.5:0.5-3.

15. The method of claim 14, wherein the diabetes medication is insulin, a sulfonylurea, a meglitinide, a biguanide, a thiazolidinedione, an alpha-glucosidase inhibitor, a DPP-4 inhibitor, or an adjunct therapy.

16. The method of claim 15, wherein the diabetes medication is metformin and the quercetin is isoquercetin.

17. The method of claim 14, wherein the composition further includes folic acid, genistein, genistin, curcumin, resveratrol, isoquercetin, luteolin, epigallocatechin gallate (EGCG), coenzyme Q10, eicosapentaenoic acid (EPA), or docosahexaenoic acid (DHA).

18. A method for treating metabolic syndrome, the method comprising administering to a subject in need thereof an effective amount of a composition that includes quercetin, vitamin B3, vitamin C, and a Bauhinia fortificata extract, wherein a weight ratio between quercetin, vitamin B3, vitamin C, and the Bauhinia fortificata extract is 1:0.02-1.5:0.2-2.5:0.5-3.

19. The method of claim 18, wherein the composition further includes folic acid, genistein, genistin, curcumin, resveratrol, isoquercetin, luteolin, epigallocatechin gallate (EGCG), coenzyme Q10, eicosapentaenoic acid (EPA), or docosahexaenoic acid (DHA).

20. A method for treating diabetes, the method comprising administering to a subject in need thereof an effective amount of a composition that includes quercetin, vitamin B3, vitamin C, a Bauhinia fortificata extract, luteolin, and epigallocatechin gallate (EGCG) as the only active ingredients, and a weight ratio between quercetin, vitamin B3, vitamin C, and the Bauhinia fortificata extract is 1:0.02-1.5:0.2-2.5:0.5-3.
